(19) United States
(12) Patent Application Publication
(10) Pub. No.: US 2013/0225497 A1
(43) Pub. Date: Aug. 29, 2013

(54) SKIN COLLAGEN PRODUCTION-PROMOTING AGENT

(71) Applicants: Ken Kato, Hokkaido (JP); Aiko Ono, Hokkaido (JP); Hiroshi Ueno, Hokkaido (JP); Noriko Ueda, Hokkaido (JP); Hiroaki Matsuyama, Hokkaido (JP)

(72) Inventors: Ken Kato, Hokkaido (JP); Aiko Ono, Hokkaido (JP); Hiroshi Ueno, Hokkaido (JP); Noriko Ueda, Hokkaido (JP); Hiroaki Matsuyama, Hokkaido (JP)

(73) Assignee: MEGMILK SNOW BRAND CO., LTD., Hokkaido (JP)

(21) Appl. No.: 13/876,889

(22) PCT Filed: Sep. 29, 2011
(86) PCT No.: PCT/JP2011/072362

§ 371 Date: May 15, 2013

(30) Foreign Application Priority Data
Sep. 30, 2010 (JP) 2010-222099

(51) Int. Cl. A61K 8/64; A23L 1/305; A23L 2/66; A61Q 19/00

(52) U.S. Cl. CPC A61K 8/64; A61Q 19/00; A23L 1/3056; A23L 2/66
USPC 514/17.2

(57) ABSTRACT

A skin collagen production-promoting agent includes a milk protein fraction having following properties (a) to (c) as an active ingredient: (a) being derived from milk, (b) containing proteins having a molecular weight determined by sodium dodecyl sulfate-polyacrylamide gel electrophoresis (SDS-PAGE) of 6000 to 150,000 daltons, and (c) containing 12 to 14 wt % of basic amino acids with respect to the constituent amino acid composition, and having a basic amino acid/acidic amino acid ratio of 0.5 to 0.7.

### TECHNICAL FIELD

The invention relates to a skin collagen production-promoting agent, a food or beverage product for promoting skin collagen production, and a cosmetic for promoting skin collagen production, which are useful in preventing rough skin , wrinkles, the loss of elasticity in the skin, or the like. More specifically, the invention relates to a skin collagen production-promoting agent that includes a milk protein fraction and/or a degradation product of the milk protein fraction as an active ingredient.

### BACKGROUND ART

In recent years, studies on the mechanism of skin have been carried out, and as the results, it has been confirmed that macroscopic causes of dry feeling of the skin and rough skin are complexly involved in the effects of sunlight (ultraviolet), drying, oxidation, and the like in addition to effects due to decrement of the metabolism with aging. It has been found that these effects caused by such factors significantly decrease the amount of collagen that are the main matrix component in the dermis. When a mechanism to keep tension or elasticity of the skin that is maintained by the collagen is destroyed by the effects of ultraviolet or the like, wrinkles or slacks of the skin increase. The collagen molecules can maintain water, so that it helps maintaining skin moisture. Therefore, the skin becomes dry and rough when collagen is destroyed by the external factors. From the above, a skin collagen production-promoting agent that is safe and can prevent wrinkles and slacks of the skin on skin by promoting biosynthesis of collagen that is one of the main components of the dermis has been desired.

A basic protein fraction contained in milk, or a degraded basic protein fraction obtained by degrading the basic protein fraction using a protease (see Patent Document 1), lactoferrin or degraded lactoferrin obtained by degrading the lactoferrin using a protease (see Patent Document 2), lactoperoxidase or degraded lactoperoxidase obtained by degrading the lactoperoxidase using a protease (see Patent Document 3), and angiogenin or degraded angiogenin obtained by degrading the angiogenin using a protease (see Patent Document 4), have been known as a substance that exhibits a promoting effect on skin collagen production.

### Prior Art Document

### Patent Document

### Patent Document 1: JP-A-2003-144095

### Patent Document 2: JP-A-2004-331564

### Patent Document 3: JP-A-2004-331565

### Patent Document 4: JP-A-2004-331566

### SUMMARY OF THE INVENTION

### Problems to be Solved by the Invention

An object of the invention is to provide a novel skin collagen production-promoting agent that can prevent dry feeling of the skin, rough skin, wrinkles and slacks of the skin, can be used as a food material which has not any problem on safety, and promotes biosynthesis of collagen. Another object of the invention is to provide a food or beverage product, or a cosmetic that includes a skin collagen production-promoting agent.

### Means for Solving the Problems

In order to achieve the above objects, the inventors of the invention have conducted extensive studies on a promoting effect on skin collagen production by using substances that is widely contained in food materials. As a result, the inventors have found out a milk protein fraction and/or a degradation product of the milk protein fraction which exhibits a promoting effect on skin collagen production. This finding has led to the completion of the present invention.

Specifically, the invention includes the followings:

(1) A skin collagen production-promoting agent including a milk protein fraction having following properties (a) to (c) as an active ingredient:

(a) being derived from milk,
(b) containing proteins having a molecular weight determined by sodium dodecyl - sulfate-polyacrylamide gel electrophoresis (SDS-PAGE) of 6000 to 150,000 daltons, and
(c) containing 12 to 14 wt % of basic amino acids with respect to the constituent amino acid composition, and having a basic amino acid/acidic amino acid ratio of 0.5 to 0.7.

(2) A skin collagen production-promoting agent including a degradation product of a milk protein fraction having following properties (a) to (c) as an active ingredient:

(a) being derived from milk,
(b) containing proteins having a molecular weight determined by sodium dodecyl sulfate-polyacrylamide gel electrophoresis (SDS-PAGE) of 6000 to 150,000 daltons, and
(c) containing 12 to 14 wt % of basic amino acids with respect to the constituent amino acid composition, and having a basic amino acid/acidic amino acid ratio of 0.5 to 0.7.

(3) A food or beverage product for promoting skin collagen production including the milk protein fraction according to (1) or the degradation product of the milk protein fraction according to (2).

(4) A cosmetic for promoting skin collagen production including the milk protein fraction according to (1) or the degradation product of the milk protein fraction according to (2).

(5) A method for promoting skin collagen production including oral administration of a milk protein fraction having following properties (a) to (c) in an amount of 10 μg/day or more per adult, or applying a composition that includes the milk protein fraction in an amount of 0.001 to 2 wt % based on a total amount of the composition:

(a) being derived from milk,
(b) containing proteins having a molecular weight determined by sodium dodecyl sulfate-polyacrylamide gel electrophoresis (SDS-PAGE) of 6000 to 150,000 daltons, and
(c) containing 12 to 14 wt % of basic amino acids with respect to the constituent amino acid composition, and having a basic amino acid/acidic amino acid ratio of 0.5 to 0.7.

(6) A method for promoting skin collagen production including oral administration of a degradation product of a milk protein fraction having following properties (a) to (c) in an amount of 10 μg/day or more per adult, or applying a composition that includes the degradation product in an amount of 0.001 to 2 wt % based on a total amount of the composition:

(a) being derived from milk,
(b) containing proteins having a molecular weight determined by sodium dodecyl sulfate-polyacrylamide gel electrophoresis (SDS-PAGE) of 6000 to 150,000 daltons, and
(c) containing 12 to 14 wt % of basic amino acids with respect to the constituent amino acid composition, and having a basic amino acid/acidic amino acid ratio of 0.5 to 0.7.

### EFFECTS OF THE INVENTION

The skin collagen production-promoting agent according to the invention promotes biosynthesis of collagen in skin through oral administration or application, and may be thus useful for preventing dry feeling of skin, rough skin, wrinkles, the loss of elasticity in the skin, and the like.

### EMBODIMENTS FOR CARRYING OUT THE INVENTION

A skin collagen production-promoting agent according to the invention includes a milk protein fraction or a degradation product of the milk protein fraction as an active ingredient, and the milk protein fraction and the degradation product absolutely satisfy the following three conditions (a) to (c):

(a) being derived from milk,
(b) containing proteins having a molecular weight determined by sodium dodecyl sulfate-polyacrylamide gel electrophoresis (SDS-PAGE) of 6000 to 150,000 daltons, and
(c) containing 12 to 14 wt % of basic amino acids with respect to the constituent amino acid composition, and having a basic amino acid/acidic amino acid ratio of 0.5 to 0.7. The milk protein fraction may be obtained by applying a milk raw material, such as skim milk, whey or the like to a cation-exchange resin, washing the cation-exchange resin with deionized water, and eluting the milk protein adhered on the cation-exchange resin by using a 0.2M sodium chloride. Note that as the salt, a potassium salt, an ammonium salt, a phosphate, an acetate, a carbonate, or the like may be used instead of sodium chloride. The milk protein fraction according to the invention may be obtained by appropriately adjusting the ionic strength of the washing solution to not more than 0.05 and the ionic strength of the eluant to 0.15 to 0.25. The milk protein fraction may also be obtained by collecting the eluted fraction, which is desalted and concentrated by using a reverse osmosis (RO) membrane, electrodialysis (ED), or the like, and optionally drying the resulting product. Examples of the reverse osmosis (RO) membrane include Desal-3 (manufactured by Desalination), HR-95 (manufactured by Dow Danmark), NTR-729HF (manufactured by Nitto Denko Corporation), and the like. Examples of an electrodialysis (ED) system include electrodialysis systems manufactured by Yuasa-Ionics Inc. and Nippon Rensui Co., Ltd.

The following methods have been known as a method of obtaining a protein fraction derived from milk; a method of obtaining the fraction by contacting milk or a raw material derived from milk with a cation exchanger, and then eluting the basic protein fraction adsorbed on the cation exchanger using an eluant having a pH of more than 5 and an ionic strength of more than 0.5 (JP-A-5-202098), a method of obtaining the fraction using an alginic acid gel (JP-A-61-246198), a method of obtaining the fraction from a whey using porous inorganic particles (JP-A-1-86839), a method of obtaining the fraction from milk using a sulfated ester compound (JP-A-63-255300), or the like. A protein fraction obtained by such a method may be used for the skin collagen production-promoting agent according to the invention. The milk protein fraction thus collected may be used after powdering by freeze-drying or the like.

The milk protein fraction which is used in the invention and exerts a promoting effect on skin collagen production contains 12 to 14 wt % of basic amino acids with respect to the constituent amino acid composition, and has a basic amino acid/acidic amino acid ratio of 0.5 to 0.7. The advantageous effects of the invention may be impaired if the content of basic amino acids or the basic amino acid/acidic amino acid ratio is outside the above range. The milk protein fraction used in the invention is a mixture of various proteins having a molecular weight of 6000 to 150,000 daltons, and has an isoelectric point over a wide range of 6 to 11.

The degradation product of the milk protein fraction has the same amino acid composition as that of the milk protein fraction. For example, a degradation product having an average molecular weight of 4000 or less may be obtained by hydrolyzing the milk protein fraction obtained by the above methods with a protease such as pepsin, trypsin, chymotrypsin or the like, and optionally further hydrolyzing with a protease such as pancreatin or the like. The degradation product of the milk protein fraction may be used after powdering by freeze-drying or the like.

As milk or a raw material derived from milk used to produce the milk protein fraction which is an active ingredient of the skin collagen production-promoting agent according to the invention, milk such as bovine milk, human milk, goat milk, ewe milk or the like may be used. Such milk may be used without additional treatment, or reconstituted milk, skim milk, whey, or the like thereof may be used.

The skin collagen production-promoting agent according to the invention exerts a promoting effect on skin collagen production through oral administration or application. In the case of oral administration of the skin collagen production-promoting agent of the present invention, the milk protein fraction or the degradation product of the milk protein fraction (i.e., active ingredient) may be orally administered as it is (without additional treatment). Of course, the fraction and the degradation product may be used after it is formulated into a powder, granule, tablet, capsule, drink, or the like according to the conventional method. In the present invention, an oral preparation such as a powder, granule, tablet, or capsule is formulated by using a vehicle such as starch, lactose, sucrose, mannitol, carboxymethylcellulose, cornstarch, or inorganic salts or the like according to the conventional method. For formulation, there may be used, in addition to the above described vehicles, a binder, disintegrator, surfactant, lubricant, fluidity promoter, colorant, flavor, or the like. More specifically, examples of the binder include starch, dextrin, powdered acacia, gelatin, hydroxypropyl starch, sodium carboxymethylcellulose, methyl cellulose, crystalline cellulose, ethyl cellulose, polyvinylpyrrolidone, for example. Examples of the disintegrator include starch, hydroxypropyl starch, carboxymethylcellulose, sodium carboxymethylcellulose, crosslinked sodium carboxymethylcellulose, crystalline cellulose, and the like. Examples of the surfactant include soybean lecithin, sucrose fatty acid ester, and the like. Examples of the lubricant include talc, wax, sucrose fatty acid ester, hydrogenated vegetable oils, and the like. Examples of the fluidity promoter include silicic anhydride, dried aluminum hydroxide, magnesium silicate, and the like.

The milk protein fraction, or the degradation product thereof may be incorporated in a nutrient supplement, a food or beverage product, or the like, without additional treatment or after being formulated. In addition, when the milk protein fraction or the degradation product thereof is incorporated in combination with an ingredient such as vitamin C that is conventionally considered to have a good effect on collagen production, further promoting effect on skin collagen production may be expected. Since the milk protein fraction or the degradation product thereof is relatively stable against heat, a material containing the milk protein fraction or the degradation product thereof can be sterilized by heating under a general normal condition.

In the case of application of the skin collagen production-promoting agent according to the invention, the skin collagen production-promoting agent may be incorporated in a known ingredient that is generally used depending on the intended use to thereby prepare various dosage forms such as a liquid formulation, a solid formulation, a semi-solid formulation, and the like. Examples of a preferable composition include an ointment, gel, cream, spray, patch, lotion, powder, and the like. For example, the skin collagen production-promoting agent according to the invention may be mixed with a hydrocarbon such as petrolatum, higher fatty acid lower alkyl ester such as stearyl alcohol or isopropyl myristate, animal oil and fat such as lanoline, polyol such as glycerin, surfactant such as glycerin fatty acid ester, or polyethyleneglycol monostearate, inorganic salt, wax, resin, water, and if necessary, preservative such as methyl parahydroxybenzoate or butyl parahydroxybenzoate, to thereby produce a cosmetic or pharmaceutical product for promoting skin collagen production.

The effective amount of the skin collagen production-promoting agent according to the invention for oral administration is appropriately determined depending on the dosage form, administration method, intended use, and the age, the weight, and the symptom of a patient to whom the skin collagen production-promoting agent is administered, and it is not constant. However, the results of animal experiments using rats revealed that, the milk protein fraction or the degradation product of the milk protein fraction must be taken in an amount of 10 μg or more per 1 kg of rat weight in order to exert the promoting effect on skin collagen production. Therefore, according to the extrapolation method, when the milk protein fraction or the degradation product of the milk protein fraction is taken in an amount of 10 μg/day or more per adult, the effect may be expected. Accordingly, the milk protein fraction or the degradation product of the milk protein fraction may be incorporated in a food or beverage product, or may be administered as a drug so that the above amount is ensured. Note that the milk protein fraction or the degradation product of the milk protein fraction may optionally be administered several times a day.

The effective amount of the skin collagen production-promoting agent according to the invention for application varies depending on the dosage form, but the milk protein fraction or the degradation product of the milk protein fraction may be incorporated in the amount of 0.001 to 2wt % on the basis of the amount of all compositions to be applied. Note that the amount of the milk protein fraction or the degradation product of the milk protein fraction may be further increased when the composition is diluted during use as a bath powder.

The invention is further described below in detail by way of reference examples, examples, and test examples, but, the following examples merely illustrate some embodiments of the invention, and should not be construed as limiting the invention.

### REFERENCE EXAMPLE 1

A milk protein fraction exhibiting a promoting effect on skin collagen production was prepared by the following method (see JP-A-2003-144095). A column having 10 cm of diameter and charged with 0.5 l (liter) of cation-exchange resin (Sulfonated Chitopearl; manufactured by Fuji Spinning Co., Ltd.) was sufficiently washed with deionized water. After applying 50 l of unsterilized skim milk through the column at a flow rate of 100 ml/min, the column was sufficiently washed with deionized water. 2.5 l of 0.05M phosphate buffer (pH 7.0) containing 0.95M sodium chloride was apllied the column to elute proteins adhered on the resin. The eluate was desalted and concentrated using a reverse osmosis (RO) membrane, and freeze-dried to obtain a powdery milk protein fraction. The above operations were repeated twice to obtain 104 g of a protein fraction. The protein fraction had an isoelectric point of 7.0 to 8.5. The content of basic amino acids in the protein fraction was 17.8%.

### Example 1

A column having 10 cm of diameter and charged with 0.5 l of cation-exchange resin (Sulfonated Chitopearl; manufactured by Fuji Spinning Co., Ltd.) was sufficiently washed with deionized water. After applying 50 l of unsterilized skim milk through the column at a flow rate of 100 ml/min, the column was sufficiently washed with deionized water. 2.5 l of 0.05M phosphate buffer (pH 7.0) containing 0.15M sodium chloride was applized the column to elute proteins adhered on the resin. The eluate was desalted and concentrated using a reverse osmosis (RO) membrane, and freeze-dried to obtain a powdery milk protein fraction. The above operations were performed ten times to obtain 24.2 g of a milk protein fraction. The milk protein fraction had a molecular weight of 6000 to 150,000 daltons and an isoelectric point of 6.0 to 11.0. The content of basic amino acids in the milk protein fraction was 12 to 14% with respect to the constituent amino acid. The milk protein fraction had a basic amino acid/acidic amino acid ratio of 0.5 to 0.7. The milk protein fraction thus obtained may be used without additional treatment as the skin collagen production-promoting agent according to the invention.

### Example 2

A column having 10 cm of diameter and charged with 0.5 l of cation-exchange resin (Sulfonated Chitopearl; manufactured by Fuji Spinning Co., Ltd.) was sufficiently washed with deionized water. After applying 50 l of unsterilized skim milk through the column at a flow rate of 100 ml/min, the column was sufficiently washed with 0.05M phosphate buffer (pH 7.0) containing 0.05M sodium chloride. 2.5 l of 0.05M phosphate buffer (pH: 7.0) containing 0.25M sodium chloride was applied the column to elute proteins adhered on the resin. The eluate was desalted and concentrated using a reverse osmosis (RO) membrane, and freeze-dried to obtain a powdery milk protein fraction. The above operations were performed five times to obtain 12.8 g of a milk protein fraction. The milk protein fraction had a molecular weight of 6000 to 150,000 daltons and an isoelectric point of 6.0 to 11.0. The content of basic amino acids in the milk protein fraction was 12 to 14% with respect to the constituent amino acid. The milk protein fraction had a basic amino acid/acidic amino acid ratio of 0.5 to 0.7. The milk protein fraction thus obtained may be used without additional treatment as the skin collagen production-promoting agent according to the invention.

### Example 3

24.2 g of the milk protein fraction obtained in Example 1 was dissolved in 10 l of distilled water. After adding pepsin (manufactured by Kanto Kagaku Co., Ltd.) at a concentration of 2%, the milk protein fraction was hydrolyzed at 37° C. for 1 hour with stirring. After neutralizing the mixture to pH 6.8 using a sodium hydroxide solution, 1% pancreatin (manufactured by Sigma) was added to the mixture. The mixture was then reacted at 37° C. for 2 hours. After completion of the reaction, the protease was inactivated by heating the mixture at 80° C. for 10 minutes to obtain 23.1 g of a degradation product of the milk protein fraction. The degradation product thus obtained may be used without additional treatment as the skin collagen production-promoting agent according to the invention.

### Example 4

12.8 g of the milk protein fraction obtained in Example 2 was dissolved in 8 l of distilled water. After adding trypsin (manufactured by Kanto Kagaku Co., Ltd.) at a concentration of 2%, the milk protein fraction was hydrolyzed at 37° C. for 1 hour with stirring. After neutralizing the mixture to pH 6.6 using sodium hydroxide solution, 1% pancreatin (manufactured by Sigma) was added to the mixture and then reacted at 37° C. for 2 hours. After completion of the reaction, the protease was inactivated by heating the mixture at 80° C. for 10 minutes to obtain 11.7 g of a degradation product of the milk protein fraction. The degradation product thus obtained may be used as the skin collagen production-promoting agent according to the invention without additional treatment.

### Test Example 1

The promoting effect on skin collagen production of the milk protein fraction obtained in Reference Example 1, the milk protein fraction obtained in Example 1, and the degradation product obtained in Example 3 was determined by animal experiments using rats. 7-week-old Wistar male rats were divided into following seven groups (n=6); a group to which physiological saline was administered (group A), a group to which the milk protein fraction obtained in Reference Example 1 was administered in an amount of 10 μg per 1 kg of weight (group B), a group to which the milk protein fraction obtained in Reference Example 1 was administered in an amount of 100 μg per 1 kg of weight (group C), a group to which the milk protein fraction obtained in Example 1 was administered in an amount of 10 μg per 1 kg of weight (group D), a group to which the milk protein fraction obtained in Example 1 was administered in an amount of 100 μg per 1 kg of weight (group E), a group to which the degradation product obtained in Example 3 was administered in an amount of 10 μg per 1 kg of weight) (group F), and a group to which the degradation product obtained in Example 3 was administered in an amount of 100 μg per 1 kg of weight (group G). Every rat was kept for 10 weeks during which the milk protein fraction or the degradation product was administered by using a sonde once a day. The amount of collagen in the skin was determined by treating the dermis of each rat in accordance with the method by Nimni et al. (see Arch. Biochem. Biophys., p. 292, 1967), and measuring the hydroxyproline content in the soluble fraction. Hydroxyproline is a specific amino acid contained only in collagen, and consitutes 10% of all amino acids of collagen. Therefore, the amount of collagen can be estimated (see Ryuji Asano et al., BioIndustry, p. 12, 2001). The results are shown in Table 1.

TABLE 1
Hydroxyproline content (μg/ml)

| | |
|---|---|
| Group A | 0.3 ± 0.1 |
| Group B | 0.5 ± 0.2* |
| Group C | 0.7 ± 0.1* |
| Group D | 1.0 ± 0.2* |
| Group E | 1.4 ± 0.3* |
| Group F | 0.9 ± 0.1* |
| Group G | 1.5 ± 0.2* |

The value shown in Table 1 indicates “mean±standard deviation” (n=6). The symbol “*” indicates that there was a significant difference as compared with the group A that is a control group (p<0.05).

As shown in Table 1, the amount of hydroxyproline in the soluble fraction after 10 weeks was significantly larger in the groups B, C, D, E, F, and G as compared with the group A. It was also found that the milk protein fraction obtained in Example 1 and the degradation product obtained in Example 3 had an effect about twice that of the milk protein fraction obtained in Reference Example 1. It was thus confirmed that the milk protein fraction or the degradation product of the milk protein fraction according to the invention has a promoting effect on skin collagen production, and is useful as a skin collagen production-promoting agent. It is revealed that the promoting effect on skin collagen production is obtained when the milk protein fraction or the degradation product of the milk protein fraction is administered in an amount of at least 10 μg per 1 kg of rat weight.

### Example 5

A beverage containing a skin collagen production-promoting agent in the composition shown in Table 2 was produced according to the conventional method. The beverage had a good flavor, the flavor did not deteriorate even after 1 year storage at ambient temperature, and there was no problem such as generation of precipitates and the like.

TABLE 2

| | |
|---|---|
| Mixed isomerized sugar | 15.0 (wt %) |
| Fruit juice | 10.0 |
| Citric acid | 0.5 |
| Skin collagen production-promoting | 0.1 |
| agent (Invention product 2) | |
| Flavor | 0.1 |
| Mineral mixture | 0.1 |
| Water | Balance |

### Example 6

Dough having the composition shown in Table 3 was prepared according to the conventional method, then formed and baked to produce biscuits containing a skin collagen production-promoting agent.

TABLE 3

| | |
|---|---|
| Flour | 50.0 (wt %) |
| Sugar | 20.0 |
| Salt | 0.5 |
| Margarine | 12.5 |
| Egg | 12.5 |
| Water | 3.5 |
| Mineral mixture | 0.8 |
| Skin collagen production-promoting | 0.2 |
| agent (Invention product 3) | |

### Example 7

A skin collagen production-promoting agent having the composition shown in table 4 was produced according to the conventional method.

TABLE 4

| Hydrous crystalline glucose | 90.5 (wt %) |
|---|---|
| Mineral mixture | 5.0 |
| Skin collagen production-promoting | 3.0 |
| agent (Invention product 1) | |
| Sugar ester | 1.0 |
| Flavor | 0.5 |

The components were mixed in the composition shown in Table 5, and emulsified at 85° C. to produce a processed cheese containing a skin collagen production-promoting agent.

TABLE 5

| Gouda cheese | 43.0 (wt %) |
|---|---|
| Cheddar cheese | 43.0 |
| Sodium citrate | 2.0 |
| Skin collagen production-promoting | 0.5 |
| agent (Invention product 4) | |
| Water | 11.5 |

### Test Example 2

The promoting effect on skin collagen production of the milk protein fraction obtained in Example 2 (Invention product 2) and the degradation product obtained in Example 4 (Invention product 4) was determined by experiments using a normal human fibroblast strain (CCD45SK (ATCCRL 1506) collected from the skin of a white female). The cells were seeded to a 24-well plate at a concentration of 4×10⁴ cells/well/0.4 ml using a modified Eagle medium (MEM) (“10-101” manufactured by Dainippon Pharmaceutical Co., Ltd.) containing 10 vol % bovine fetal serum (hereinafter abbreviated as FBS), and cultured at 37° C. for 24 hours under 5% carbon dioxide and saturated water vapor, and then the medium was replaced with an MEM containing 0.6 vol % FBS. Thereafter, the milk protein fraction obtained in Example 2 (Invention product 2) and the degradation product obtained in Example 4 (Invention product 4) were added to each well at a concentration of 0.1 vol %, and the cells were cultured for 24 hours. After that, β-aminopropionitrile and tritiated L-proline at concentration of 50 μg/ml and 1 μCi/ml, respectively, and the cells were cultured for additional 24 hours to obtain a culture solution. A collagen fraction was fractionated from the culture solution in accordance with the method by Webster et al. (see Analytical Biochemistry, p. 220, 1979), and the amount of radioactivity that had been taken in the collagen fraction was measured. Note that similar test was performed without adding the milk protein fraction or the degradation product of the milk protein fraction as a control.

The results are shown in Table 6.

TABLE 6

| | Collagen |
|---|---|
| | production (%) |
| Control | 100 ± 5 |
| Milk protein fraction | 259 ± 11* |
| (Invention product 2) | |
| Degradation product of milk protein | 247 ± 9* |
| fraction (Invention product 4) | |

The value shown in Table 6 indicates “mean±standard deviation (n=6)”. The symbol “*” indicates that there was a significant difference as compared with the control group (p<0.05).

As shown in Table 6, the promoting effect on skin collagen production obtained in the groups to which the milk protein fraction or the degradation product of the milk protein fraction was added was equal to or more than twice as compared with that of the group (Control) to which the milk protein fraction or the degradation product of the milk protein fraction was not added. The results revealed that the milk protein fraction and the degradation product of the milk protein fraction according to the invention respectively affect skin fibroblasts, and have a promoting effect on skin collagen production, and are useful as a skin collagen production-promoting agent.

### Example 8

A lotion having the composition shown in Table 7 was produced according to the conventional method.

TABLE 7

| | |
|---|---|
| Glycerin | 3.0 (wt %) |
| 1,3-Butylene glycol | 3.0 |
| Polyoxyethylene sorbitan monooleate | 0.5 |
| Methyl parahydroxybenzoate | 0.15 |
| Citric acid | 0.1 |
| Sodium citrate | 1.0 |
| Flavor | 0.05 |
| Skin collagen production-promoting | 0.05 |
| agent (Invention product 3) | |
| Purified water | Balance |

### Example 9

Cream having the composition shown in Table 8 was produced according to the conventional method.

TABLE 8

| | |
|---|---|
| Liquid paraffin | 5.0 (wt %) |
| White beeswax | 4.0 |
| Cetanol | 3.0 |
| Squalane | 10.0 |
| Lanolin | 2.0 |
| Stearic acid | 1.0 |
| Polyoxyethylene sorbitan monooleate | 1.5 |
| Glyceryl monostearate | 3.0 |
| 1,3-Butylene glycol | 6.0 |
| Methyl parahydroxybenzoate | 1.5 |
| Flavor | 0.1 |
| Skin collagen production-promoting | 0.5 |
| agent (Invention product 2) | |
| Purified water | Balance |

### Test Example 3

A clinical test was performed using the skin lotion obtained in Example 8 and the cream obtained in Example 9. As a lotion and cream for comparison, there were used lotion and cream having the same compositions as those in Example 8 and 9, except that the milk protein fraction or the degradation product of the milk protein fraction was not added. Twenty adult females having face with dry skin, slacks and fine wrinkles of the skin were divided at random into two groups each having 10 subjects (groups A and B), and twenty adult females having hands and fingers with rough skin were divided at random into two groups each having 10 subjects (groups C and D). 2 g of the lotion according to the invention, 2 g of the lotion for comparison, 2 g of the cream according to the invention, and 2 g of the cream for comparison were applied to the faces of group A, the faces of group B, the hands and fingers of group C, and the hands and fmgers of group D, respectively, in the same way as usual twice a day for 10 days. The results are shown in Table 9.

TABLE 9

| | Dry feeling | Rough skin | Wrinkles | Slacks |
|---|---|---|---|---|
| Group A | ++ | ++ | + | ++ |
| Group B | ± | ± | ± | ± |
| Group C | + | ++ | Not measured | Not measured |
| Group D | ± | ± | Not measured | Not measured |

++; A significant effect was observed after 10 days
+; A effect was observed after 10 days
±; Any effect was not observed after 10 days

As shown in Table 9, it was revealed that the lotion of the invention significantly improved dry feeling of the skin, rough skin, and the like, and exhibited an excellent promoting effect on skin collagen production as compared with the comparative lotion. It was also revealed that the cream of the invention significantly improved dry feeling of the skin, rough skin, and the like, and had an effect suppressing spontaneous exacerbation, such as rough skin as compared with the comparative cream.

1. A skin collagen production-promoting agent comprising a milk protein fraction having following properties (a) to (c) as an active ingredient:
(a) being derived from milk,
(b) containing proteins having a molecular weight determined by sodium dodecyl sulfate-polyacrylamide gel electrophoresis (SDS-PAGE) of 6000 to 150,000 daltons, and
(c) containing 12 to 14 wt % of basic amino acids with respect to the constituent amino acid composition, and having a basic amino acid/acidic amino acid ratio of 0.5 to 0.7.

2. A skin collagen production-promoting agent comprising a degradation product of a milk protein fraction having following properties (a) to (c) as an active ingredient:
(a) being derived from milk,
(b) containing proteins having a molecular weight determined by sodium dodecyl sulfate-polyacrylamide gel electrophoresis (SDS-PAGE) of 6000 to 150,000 daltons, and
(c) containing 12 to 14 wt % of basic amino acids with respect to the constituent amino acid composition, and having a basic amino acid/acidic amino acid ratio of 0.5 to 0.7.

3. A food or beverage product for promoting skin collagen production comprising the milk protein fraction according to claim 1.

4. A cosmetic for promoting skin collagen production comprising the milk protein fraction according to claim 1.

5. A method for promoting skin collagen production comprising oral administration of a milk protein fraction having following properties (a) to (c) in an amount of 10 μg/day or more per adult, or applying a composition that includes the milk protein fraction in an amount of 0.001 to 2 wt % based on a total amount of the composition:
(a) being derived from milk,
(b) containing proteins having a molecular weight determined by sodium dodecyl sulfate-polyacrylamide gel electrophoresis (SDS-PAGE) of 6000 to 150,000 daltons, and
(c) containing 12 to 14 wt % of basic amino acids with respect to the constituent amino acid composition, and having a basic amino acid/acidic amino acid ratio of 0.5 to 0.7.

6. A method for promoting skin collagen production comprising oral administration of a degradation product of a milk protein fraction having following properties (a) to (c) in an amount of 10 μg/day or more per adult, or applying a composition that includes the degradation product in an amount of 0.001 to 2 wt % based on a total amount of the composition:
(a) being derived from milk,
(b) containing proteins having a molecular weight determined by sodium dodecyl sulfate-polyacrylamide gel electrophoresis (SDS-PAGE) of 6000 to 150,000 daltons, and
(c) containing 12 to 14 wt % of basic amino acids with respect to the constituent amino acid composition, and having a basic amino acid/acidic amino acid ratio of 0.5 to 0.7.

7. A food or beverage product for promoting skin collagen production comprising the degradation product according to claim 2.

8. A cosmetic for promoting skin collagen production comprising the degradation product according to claim 2.
